(19) United States
(12) Patent Application Publication
(10) Pub. No.: US 2024/0374496 A1
(43) Pub. Date: Nov. 14, 2024

(54) COMPOSITION FOR CARING FOR KERATIN MATERIALS

(71) Applicant: L'OREAL, Paris (FR)

(72) Inventors: Hongling XU, Shanghai (CN); Yangdong CHEN, Shanghai (CN); Yirui ZHU, Shanghai (CN)

(73) Assignee: L'OREAL, Paris (FR)

(21) Appl. No.: 18/291,956

(22) PCT Filed: Aug. 31, 2021
(86) PCT No.: PCT/CN2021/115542

§ 371 Date: Jan. 25, 2024

(51) Int. Cl. A61K 8/60; A61K 8/06; A61K 8/37; A61Q 19/00

(52) U.S. Cl. CPC A61K 8/602; A61K 8/062; A61K 8/375; A61Q 19/00

(57) ABSTRACT

A composition in the form of an oil-in-water emulsion for caring for keratin materials comprising: (i) at least one cosmetic active compound selected from C-glycosides (ii); at least one anionic surfactant; (iii) at least one non-ionic surfactant selected from alkoxylated alcohols; (iv) at least one non-ionic surfactant selected from fatty acid esters of polyglycerol. And a non-therapeutic method for caring for keration materials, applying said composition to the keratin materials.

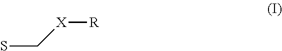

### TECHNICAL FIELD

The present invention relates to a cosmetic composition. In particular, the present invention relates to a composition for caring for keratin materials. The present invention also relates to a non-therapeutic method for caring for keratin materials.

### BACKGROUND ART

The skin is the protective barrier for the human body. It protects the interior of the body from physical injury (such as trauma) and biological injury (such as bacteria, viruses or fungi). The epidermis is a keratinized stratified pavimentous epithelium. Its mean thickness ranges from 60 to 100 μm and may reach 600 to 700 μm on the sole of the feet and the palm of the hands. It consists mainly of keratinocytes, but also other cells, and rests on a basal membrane that separates it from the dermis.

During the menopause, the skin undergoes changes in all its compartments, i.e. dermal and epidermal.

The main changes concern the dermis and are a decrease in the collagen content and in the thickness of the dermis. In menopausal women, this results in thinning of the skin and/or of the mucous membranes. Women then experience a sensation of “dry skin” or of taut skin and an accentuation of the surface wrinkles and fine lines is observed. The skin has a rough feel. Finally, the skin shows decreased suppleness.

The main changes concerning the epidermis are a decrease in keratinocyte differentiation, resulting in a deficit in the proteins matrix of the cornified cell, an increase in metalloproteinases, which are proteases that degrade the extracellular matrix and that participate in ageing of the skin, and also a decrease in the synthesis of various glycosaminoglycans.

The development of formulations dedicated to caring for and/or making up the skin and/or lips, is permanent.

A wide variety of cosmetic compositions have been used to care for the skin.

There are many cosmetic products on the market, some of them cannot be stable when subjected to a low temperature (such as at −20° C.). For example, some transparent emulsions will become cloudy after being still for one day, which is visually unpleasant.

Thus, there is still a need to formulate a composition for caring for the skin, which can deliver certain cosmetic active ingredient, which is stable even being still for several cycles from −20° C. to 20° C. per 24 hours for an extended period of time.

### SUMMARY OF THE INVENTION

The inventors have now discovered that it is possible to formulate compositions for caring for the skin, which can deliver hydroxypropyl tetrahydropyrantriol or the like and remain stable even being still for several cycles from −20° C. to 20° C. per 24 hours for an extended period of time.

Accordingly, in a first aspect, the present invention provides a composition in the form of an oil-in-water emulsion for caring for keratin materials comprising:
(i) at least one cosmetic active compound selected from C-glycosides of formula (I)

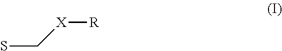

in which:
R represents a saturated C₁ to C₁₀, in particular C₁ to C₄, alkyl radical which can optionally be substituted by at least one radical chosen from OH, COOH or COOR″₂, with R″₂ being a saturated C₁-C₄ alkyl radical,
S represents a monosaccharide or a polysaccharide comprising up to 20 sugar units, in particular up to 6 sugar units, in pyranose and/or furanose form and of the L and/or D series, it being possible for the said monosaccharide or polysaccharide to be substituted by a hydroxyl group which is necessarily free and optionally one or more optionally protected amine functional group(s), and
X represents a radical chosen from the —CO—, —CH(OH)—, —CH(NH₂)—, —CH(NHCH₂CH₂CH₂OH)—, —CH(NHPh)— and —CH(CH₃)— groups and in particular a —CO—, —CH(OH)— or —CH(NH₂)— radical and more particularly a —CH(OH)— radical,
the S—CH₂—X bond represents a bond of C-anomeric nature, which can be α or β,
and also their physiologically acceptable salts, their solvates, such as the hydrates, and their optical and geometrical isomers;
(ii) at least one anionic surfactant;
(iii) at least one non-ionic surfactant selected from alkoxylated alcohol; and
(iv) at least one non-ionic surfactant selected from fatty acid esters of polyglycerol.

The composition of the present invention is in the form of an oil-in-water emulsion. Thus, said composition comprises a continuous aqueous phase and a dispersed oily phase.

Meanwhile, the composition of the present invention is transparent.

In a second aspect, the present invention provides a non-therapeutic method for caring for keratin materials, comprising applying the composition according to the first aspect of the present invention to the keratin materials.

The composition of the present invention can deliver hydroxypropyl tetrahydropyrantriol or the like and remain stable even being still for several cycles from −20° C. to 20° C. per 24 hours for an extended period of time, for example 10 days.

Other advantages of the present invention will emerge more clearly on reading the description and the examples that follow.

### DETAILED DESCRIPTION OF THE INVENTION

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “ranging from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values which are capable of being changed according to the desired purpose as required.

The term “transparent” refers to the characteristic of a composition to permit the passage of light and also allow for objects to be distinguished through a thickness of 1 cm of the composition.

For the purposes of the present invention, the term “keratin materials” is intended to cover human skin, mucous membranes such as the lips. Facial skin is most particularly considered according to the present invention.

All percentages in the present invention refer to weight percentage, unless otherwise specified.

### C-Glycosides of Formula (I)

According to the first aspect, the composition of the present invention comprises at least one cosmetic active compound selected from C-glycosides of formula (I):

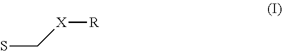

in which:
R represents a saturated C₁ to C₁₀, in particular C₁ to C₄, alkyl radical which can optionally be substituted by at least one radical chosen from OH, COOH or COOR″₂, with R″₂ being a saturated C₁-C₄ alkyl radical,
S represents a monosaccharide or a polysaccharide comprising up to 20 sugar units, in particular up to 6 sugar units, in pyranose and/or furanose form and of the L and/or D series, it being possible for the said monosaccharide or polysaccharide to be substituted by a hydroxyl group which is necessarily free and optionally one or more optionally protected amine functional group(s), and
X represents a radical chosen from the —CO—, —CH(OH)—, —CH(NH₂)—, —CH(NHCH₂CH₂CH₂OH)—, —CH(NHPh)— and —CH(CH₃)— groups and in particular a —CO—, —CH(OH)— or —CH(NH₂)— radical and more particularly a —CH(OH)— radical,
the S—CH₂—X bond represents a bond of C-anomeric nature, which can be α or β, and also their physiologically acceptable salts, their solvates, such as the hydrates, and their optical and geometrical isomers.

The C-glycosides of formula (I) of use for the implementation of the invention are in particular those for which R denotes a linear C₁ to C₆, in particular C₁ to C₄, preferentially C₁ to C₂, alkyl radical and more preferably a methyl radical.

Mention may in particular be made, among the alkyl groups suitable for the implementation of the invention, of the methyl, ethyl, isopropyl, n-propyl, n-butyl, t-butyl, isobutyl, sec-butyl, pentyl, n-hexyl, cyclopropyl, cyclopentyl or cyclohexyl groups.

According to one embodiment of the invention, use may be made of a C-glycoside compound corresponding to the formula (I) for which S can represent a monosaccharide or a polysaccharide comprising up to 6 sugar units, in pyranose and/or furanose form and of L and/or D series, the said mono- or polysaccharide exhibiting at least one necessarily free hydroxyl functional group and/or optionally one or more necessarily protected amine functional groups, X and R otherwise retaining all of the definitions given above.

Advantageously, a monosaccharide of the invention can be chosen from D-glucose, D-galactose, D-mannose, D-xylose, D-lyxose or L-fucose, L-arabinose, L-rhamnose, D-glucuronic acid, D-galacturonic acid, D-iduronic acid, N-acetyl-D-glucosamine or N-acetyl-D-galactosamine and advantageously denotes D-glucose, D-xylose, N-acetyl-D-glucosamine or L-fucose and in particular D-xylose.

More particularly, a polysaccharide of the invention comprising up to 6 sugar units can be chosen from D-maltose, D-lactose, D-cellobiose, D-maltotriose, a disaccharide combining a uronic acid chosen from D-iduronic acid or D-glucuronic acid with a hexosamine chosen from D-galactosamine, D-glucosamine, N-acetyl-D-galactosamine or N-acetyl-D-glucosamine, an oligosaccharide comprising at least one xylose which can advantageously be chosen from xylobiose, methyl-β-xylobioside, xylotriose, xylotetraose, xylopentaose and xylohexaose and in particular xylobiose, which is composed of two xylose molecules linked via a 1-4 bond.

More particularly, S can represent a monosaccharide chosen from D-glucose, D-xylose, L-fucose, D-galactose or D-maltose and in particular D-xylose.

Preferably, use is made of a C-glycoside derivative of formula (I) for which:
R denotes an unsubstituted linear C₁-C₄, in particular C₁-C₂, alkyl radical, especially a methyl radical;
S represents a monosaccharide as described above and chosen in particular from D-glucose, D-xylose, N-acetyl-D-glucosamine or L-fucose, and in particular D-xylose;
X represents a group chosen from —CO—, —CH(OH)— or —CH(NH₂)— and preferably a —CH(OH)— group.

The acceptable salts of the compounds described in the present invention comprise conventional non-toxic salts of the said compounds, such as those formed from organic or inorganic acids. Mention may be made, by way of example, of the salts of inorganic acids, such as sulfuric acid, hydrochloric acid. Mention may also be made of the salts of organic acids, which can comprise one or more carboxylic, sulfonic or phosphonic acid groups. Mention may in particular be made of propionic acid, acetic acid, terephthalic acid, citric acid and tartaric acid.

When the compound of formula (I) comprises an acid group, neutralization of the acid group(s) can be carried out with an inorganic base, such as LiOH, NaOH, KOH, Ca(OH)₂, NH₄OH, Mg(OH)₂ or Zn(OH)₂, or with an organic base, such as a primary, secondary or tertiary alkylamine, for example triethylamine or butylamine. This primary, secondary or tertiary alkylamine can comprise one or more nitrogen and/or oxygen atoms and can thus comprise, for example, one or more alcohol functional groups; mention may in particular be made of 2-amino-2-methylpropanol, triethanolamine, 2-(dimethylamino) propanol or 2-amino-2-(hydroxymethyl)-1,3-propanediol. Mention may also be made of lysine or 3-(dimethylamino)propylamine.

The solvates which are acceptable for the compounds described in the present invention comprise conventional solvates, such as those formed during the final stage of preparation of the said compounds due to the presence of solvents. Mention may be made, by way of example, of the solvates due to the presence of water or of linear or branched alcohols, such as ethanol or isopropanol.

Of course, according to the invention, a C-glycoside derivative corresponding to the formula (I) can be used alone or as a mixture with other C-glycoside derivatives and in any proportion.

A C-glycoside derivative which is suitable for the invention can in particular be obtained by the synthetic method described in the document WO 02/051828.

Mention may in particular be made, by way of non-limiting illustration of the C-glycoside compounds which are particularly suitable for the invention, of the following compounds:
C-β-D-xylopyranoside-n-propane-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxypropane,
1-(C-α-D-glucopyranosyl)-2-hydroxypropane,
1-(C-β-D-galactopyranosyl)-2-hydroxypropane,
1-(C-α-D-galactopyranosyl)-2-hydroxypropane,
1-(C-β-D-fucofuranosyl)propan-2-one,
1-(C-α-D-fucofuranosyl)propan-2-one,
1-(C-β-L-fucofuranosyl)propan-2-one,
1-(C-α-L-fucofuranosyl)propan-2-one,
C-β-D-maltopyranoside-n-propane-2-one,
C-α-D-maltopyranoside-n-propan-2-one,
C-β-D-maltopyranoside-2-hydroxypropane,
C-α-D-maltopyranoside-2-hydroxypropane, their isomers and their mixtures.

According to one embodiment, C-β-D-xylopyranoside-2-hydroxypropane or C-α-D-xylopyranoside-2-hydroxypropane and better still C-β-D-xylopyranoside-2-hydroxypropane can advantageously be used for the preparation of a composition according to the invention.

According to a specific embodiment, the C-glycoside compound can be C-β-D-xylopyranoside-2-hydroxypropane (or hydroxypropyl tetrahydropyrantriol) provided in the form of a solution containing 35% by weight of active material in a water/propylene glycol (60/40 by weight) mixture.

Advantageously, the cosmetic active compound selected from C-glycosides of formula (I) is present in the composition in an amount ranging from 0.5 wt. % to 15 wt. %, preferably from 0.6 wt. % to 13 wt. %, preferably from 0.6 wt. % to 10 wt. %, relative to the total weight of the composition.

### Anionic Surfactants

According to the first aspect, the composition of the present invention comprises at least one anionic surfactant.

Non-limiting anionic surfactant(s) that may be used in the present invention are also selected from the group comprising, alkylamido ether sulfates, alkylaryl polyether sulfates, monoglyceride sulfates, sulfonates, such as alkylsulfonates, alkylamide sulfonates, alkylarylsulfonates, alpha-olefin sulfonates, paraffin sulfonates, sulfosuccinates, alkylsulfosuccinates, alkyl ether sulfosuccinates, alkylamide sulfosuccinates, alkyl sulfoacetates, acylsarcosinates, acylglutamates, alkylsulfosuccinamates, N-acyl N-methyltaurates, N-acylisethionates, N-acyltaurates, salts of alkyl monoesters and polyglycoside-polycarboxylic acids, acyllactylates, mixed esters of organic acids with glycerol, such as glyceryl stearate citrate and as glyceryl stearate lactate, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

The anionic surfactant may also be chosen in particular from anionic derivatives of proteins of vegetable origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, anionic derivatives of alkyl poly glucosides, and their mixtures.

1) Anionic derivatives of proteins of vegetable origin are protein hydrolysates comprising a hydrophobic group, it being possible for the said hydrophobic group to be naturally present in the protein or to be added by reaction of the protein and/or of the protein hydrolysate with a hydrophobic compound. The proteins are of vegetable origin or derived from silk, and the hydrophobic group can in particular be a fatty chain, for example an alkyl chain comprising from 10 to 22 carbon atoms. Mention may more particularly be made, as anionic derivatives of proteins of vegetable origin, of apple, wheat, soybean or oat protein hydrolysates comprising an alkyl chain having from 10 to 22 carbon atoms, and their salts. The alkyl chain can in particular be a lauryl chain and the salt can be a sodium, potassium and/or ammonium salt.

2) Mention may be made, as phosphates and alkyl phosphates, for example, of monoalkyl phosphates and dialkyl phosphates, such as lauryl monophosphate, such as the product sold under the name MAP20® by Kao Chemicals, the potassium salt of dodecyl phosphate, the mixture of mono-and diesters (predominantly diester), for example sold under the name Crafol AP-31® by Cognis, the mixture of octyl phosphate monoester and diester, such as the product sold under the name Crafol AP-20® by Cognis, the mixture of ethoxylated (7 mol of EO) 2-butyloctyl phosphate monoester and diester, such as the product sold under the name Isofol 12 7 EO-Phosphate Ester® by Condea, the potassium or triethanolamine salt of mono (C12-C13) alkyl phosphate, such as the products sold under the references Arlatone MAP230K-40® and Arlatone MAP 230T-60® by Uniqema, potassium lauryl phosphate, such as the product sold under the name Dermalcare MAP XC-99/09R) by Rhodia Chimie, and potassium cetyl phosphate, for example, sold under the name Arlatone MAP 160K by Uniqema.

3) Mention may be made, as carboxylates, of:
amido ether carboxylates (AEC), such as sodium lauryl amido ether carboxylate (3 EO), for example, sold under the name Akypo Foam 30® by Kao Chemicals;
polyoxyethylenated carboxylic acid salts, such as oxyethylenated (6 EO) sodium lauryl ether carboxylate (65/25/10 C12-C14-C16), for example, sold under the name Akypo Soft 45 NV® by Kao Chemicals, polyoxyethylenated and carboxymethylated fatty acids originating from olive oil, such as the one sold under the name Olivem 400® by Biologia E Tecnologia, or oxyethylenated (6 EO) sodium tridecyl ether carboxylate, for example, sold under the name Nikkol ECTD-6NEX® by Nikkol; and
salts of fatty acids (soaps) having a C6 to C22 alkyl chain which are neutralized with an organic or inorganic base, such as potassium hydroxide, sodium hydroxide, triethanolamine, N-methylglucamine, lysine and arginine.

4) Mention may in particular be made, as amino acid derivatives, of alkali salts of amino acids, such as:
sarcosinates, such as sodium lauroyl sarcosinate, for example, sold under the name Sarkosyl NL 97® by Ciba or sold under the name Oramix L 30® by Seppic, sodium myristoyl sarcosinate, such as the product sold under the name Nikkol Sarcosinate MN® by Nikkol, or sodium palmitoyl sarcosinate, sold under the name Nikkol Sarcosinate PN® by Nikkol;
alaninates, such as sodium N-lauroyl-N-methyl amidopropionate, for example, sold under the name Sodium Nikkol Alaninate LN 30® by Nikkol or sold under the name Alanone ALE® by Kawaken, or triethanolamine N-lauroyl-N-methylalanine, for example, sold under the name Alanone ALTA® by Kawaken;
glutamates, such as triethanolamine monococoyl glutamate, such as the product sold under the name Acylglutamate CT-12® by Ajinomoto, triethanolamine lauroyl glutamate, for example, such as the product sold under the name Acylglutamate LT-12® by Ajinomoto, and sodium stearoyl glutamate;
aspartates, such as the mixture of triethanolamine N-lauroyl aspartate and triethanolamine N-myristoyl aspartate, for example, sold under the name Asparack® by Mitsubishi;
glycine derivatives (glycinates), such as sodium N-cocoyl glycinate, for example, sold under the names Amilite GCS-12® and Amilite GCK 12 by Ajinomoto;
citrates, such as the citric monoester of oxyethylenated (9 mol) coco alcohols, sold under the name Witconol EC 1 129 by Goldschmidt; and
galacturonates, such as sodium dodecyl D-galactoside uronate, sold by Soliance.

5) Mention may be made, as sulphosuccinates, for example, of oxyethylenated (3 EO) lauryl (70/30 C12/C14) alcohol monosulphosuccinate, such as the product sold under the names Setacin 103 Special® and Rewopol SB-FA 30 K 4® by Witco, the disodium salt of a hemisulphosuccinate of C12-C14 alcohols, for example, sold under the name Setacin F Special Paste® by Zschimmer Schwarz, oxyethylenated (2 EO) disodium oleamidosulphosuccinate, such as the product sold under the name Standapol SH 135® by Cognis, oxyethylenated (5 EO) lauramide monosulphosuccinate, for example, sold under the name Lebon A-5000® by Sanyo, the disodium salt of oxyethylenated (10 EO) lauryl citrate monosulphosuccinate, for example, sold under the name Rewopol SBCS 50® by Witco, or ricinoleic monoethanolamide monosulphosuccinate, for example, sold under the name Rewoderm S 1333® by Witco. Use may also be made of polydimethylsiloxane sulphosuccinates, such as disodium PEG-12 dimethicone sulphosuccinate, for example, sold under the name Mackanate-DC 30 by Macintyre.

6) Mention may be made, as alkyl sulphates, for example, of triethanolamine lauryl sulphate (CTFA name: TEA lauryl sulphate), such as the product sold by Fluntsman under the name Empicol TL40 FL or the product sold by Cognis under the name Texapon T42, which products are at 40% in aqueous solution. Mention may also be made of ammonium lauryl sulphate (CTFA name: ammonium lauryl Sulphate), such as the product sold by Huntsman under the name Empicol AL 30FL, which is at 30% in aqueous Solution.

7) Mention may be made, as alkyl ether sulphates, for example, of sodium lauryl ether sulphate (CTFA name: sodium laureth sulphate), such as that sold under the names Texapon N40 and Texapon AOS 225 UP by Cognis, or ammonium lauryl ether sulphate (CTFA name: ammonium laureth sulphate), such as that sold under the name Standapol EA-2 by Cognis.

8) Mention may be made, as sulphonates, for example, of a-olefinsulphonates, such as sodium a-olefin sulphonate (C14-C16), such as the product sold under the name Bio-Terge AS-40® by Stepan, sold under the names Witconate AOS Protege® and Sulframine AOS PH 12® by Witco or sold under the name Bio-Terge AS-40 CG® by Stepan, secondary sodium olefinsulphonate, for example, sold under the name Hostapur SAS 30® by Clariant; or linear alkylarylsulphonates, such as sodium xylenesulphonate, for example, sold under the names Manrosol SXS30®, Manrosol SXS40® and Manrosol SXS93® by Manro.

9) Mention may be made, as isethionates, of acylisethionates, such as Sodium cocoylisethionate, such as the product sold under the name Jordapon CI P® by Jordan.

10) Mention may be made, as taurates, of the sodium salt of palm kernel oil methyltaurate, for example, sold under the name Hostapon CT Pate® by Clariant; N-acyl-N-methyl taurates, such as Sodium N-cocoyl-N-methyltaurate, for example, sold under the name Hostapon LT-SF® by Clariant or sold under the name Nikkol CMT-30-T® by Nikkol, or sodium palmitoyl methyltaurate, such as the one sold under the name Nikkol PMT® by Nikkol, sodium methyl stearoyl taurate, such as Nikkol SMT® by Nikkol. The preferred is sodium methyl stearoyl taurate.

11) The anionic derivatives of alkyl polyglucosides can in particular be citrates, tartrates, sulphosuccinates, carbonates and glycerol ethers obtained from alkyl polyglucosides. Mention may be made, for example, of the sodium salt of cocoylpolyglucoside (1, 4) tartaric ester, for example, sold under the name Eucarol AGE-ET® by Cesalpinia, the disodium salt of cocoylpolyglucoside (1,4) sulphosuccinic ester, such as the product sold under the name Essai 512 MP® by Seppic, or the sodium salt of cocoylpolyglucoside (1,4) citric ester, for example, sold under the name Eucarol AGE-EC® by Cesalpinia.

In a preferred embodiment, the at least one anionic surfactant is select from taurates, preferably from sodium salt of palm kernel oil methyltaurate, sodium N-cocoyl-N-methyltaurate, sodium palmitoyl methyltaurate, sodium methyl stearoyl taurate, and mixtures thereof.

Advantageously, the anionic surfactant is present in the composition in an amount ranging from 0.05 wt. % to 2 wt. %, preferably from 0.08 wt. % to 1 wt. %, more preferably from 0.08 wt. % to 0.5 wt. %, relative to the total weight of the composition.

### Fatty Acid Esters of Polyglycerol

According to the first aspect, the composition of the present invention comprises at least one surfactant selected from fatty acid esters of polyglycerol.

Preferably, the fatty acid ester of polyglycerol (also knowns as polyglycerol fatty acid ester, PGFE) suitable for the present invention is selected from compounds of formula (II),

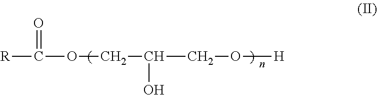

wherein:

R represents a linear or branched C₅-C₃₀ alkyl or alkenyl;

n is an integer ranging from 5 to 30.

Preferably, for the purpose of the present invention, in the formula (II),

R represents a linear or branched C₇-C₁₇ alkyl or alkenyl,

n is an integer ranging from 5 to 20, preferably from 5 to 16.

Examples of suitable PGFE surfactants are Polyglyceryl-10 Caprate, Polyglyceryl-10 Laurate, Polyglyceryl-10 Dilaurate, Polyglyceryl-10 Myristate, Polyglyceryl-10 Dimyristate, Polyglyceryl-10 Oleate, Polyglyceryl-10 Dioleate, Polyglyceryl-10 stearate, Polyglyceryl-10 Isostearate, Polyglyceryl-10 Diisostearate, Polyglyceryl-5 Laurate, Polyglyceryl-5 Dilaurate, Polyglyceryl-5 Myristate, Polyglyceryl-5 Trimyristate, Polyglyceryl-5 Oleate, or Polyglyceryl-5 Dioleate, Polyglyceryl-5 Stearate.

More particularly, the fatty acid esters of polyglycerol suitable for the present invention are compounds selected from the group consisting of Polyglyceryl-5 laurate, Polyglyceryl-5 Myristate, Polyglyceryl-5 Oleate, Polyglyceryl-5 Stearate, Polyglyceryl-10 Caprate, Polyglyceryl-10 Laurate, Polyglyceryl-10 Myristate, Polyglyceryl-10 Oleate, Polyglyceryl-10 stearate, Polyglyceryl-10 Isostearate, or a mixture thereof.

These compounds are commercially available. For example, mentions can be made to the compounds sold by the company Nikkol under the trade names: Sunsoft A-12E or Sunsoft A-121E-C (Polyglyceryl-5 laurate), Sunsoft A-14E (Polyglyceryl-5 Myristate), Sunsoft A-17E (Polyglyceryl-5 Oleate), Sunsoft A-18E (Polyglyceryl-5 Stearate), Sunsoft Q-10Y (Polyglyceryl-10 Caprate), Sunsoft Q-12Y (Polyglyceryl-10 Laurate), Sunsoft Q-14S (Polyglyceryl-10 Myristate), Nikkol Decaglyn 1-OV (Polyglyceryl-10 Oleate), and Sunsoft Q-18Y (Polyglyceryl-10 stearate).

In a particular embodiment, Polyglyceryl-5 laurate is used in the composition of the present invention.

Advantageously, the nonionic surfactant selected from fatty acid esters of polyglycerol is present in the composition in an amount ranging from 0.1 wt. % to 5 wt. %, preferably from 0.2 wt. % to 3 wt. %, more preferably from 0.25 wt. % to 2 wt. %, relative to the total weight of the composition.

### Alkoxylated Fatty Alcohols

According to the first aspect, the composition of the present invention comprises at least one nonionic surfactant selected from alkoxylated fatty alcohols.

Alkoxylated fatty alcohols, which are ethers formed from the reaction of a fatty alcohol with an alkylene oxide, generally ethylene oxide and/or propylene oxide, e.g., ethoxylated fatty alcohols which are adducts of fatty alcohols and polyethylene oxide.

Advantageously, the alkoxylated fatty alcohol is selected from ethoxylated/propoxylated alcohols, for example, ethoxylated/propoxylated C8-C30 alcohols, preferably ethoxylated/propoxylated C8-C24 alcohols, more preferably ethoxylated/propoxylated C8-C24 alcohols with 1-50 ethylene oxide units and 1-50 propylene oxide units.

Specific examples of ethoxylated/propoxylated alcohols that may be suitable for use in the present invention include PPG-1 Beheneth-15, PPG-12 Capryleth-18, PPG-2-Ceteareth-9, PPG-4-Ceteareth-12, PPG-10-Ceteareth-20, PPG-1-Ceteth-1, PPG-1-Ceteth-5, PPG-1-Ceteth-10, PPG-1-Ceteth-20, PPG-2-Ceteth-1, PPG-2-Ceteth-5, PPG-2-Ceteth-10, PPG-2-Ceteth-20, PPG-4-Ceteth-1, PPG-4-Ceteth-5, PPG-4-Ceteth-10, PPG-4-Ceteth-20, PPG-5-Ceteth-20, PPG-8-Ceteth-1, PPG-8-Ceteth-2, PPG-8-Ceteth-5, PPG-8-Ceteth-10, PPG-8-Ceteth-20, PPG-2 C12-13 Pareth-8, PPG-2 C12-15 Pareth-6, PPG-4 C13-15 Pareth-15, PPG-5 C9-15 Pareth-6, PPG-6 C9-11 Pareth-5, PPG-6 C12-15 Pareth-12, PPG-6 C12-18 Pareth-11, PPG-3 C12-14 Sec-Pareth-7, PPG-4 C12-14 Sec-Pareth-5, PPG-5 C12-14 Sec-Pareth-7, PPG-5 C12-14 Sec-Pareth-9, PPG-1-Deceth-6, PPG-2-Deceth-3, PPG-2-Deceth-5, PPG-2-Deceth-7, PPG-2-Deceth-10, PPG-2-Deceth-12, PPG-2-Deceth-15, PPG-2-Deceth-20, PPG-2-Deceth-30, PPG-2-Deceth-40, PPG-2-Deceth-50, PPG-2-Deceth-60, PPG-4-Deceth-4, PPG-4-Deceth-6, PPG-6-Deceth-4, PPG-6-Deceth-9, PPG-8-Deceth-6, PPG-14-Deceth-6, PPG-6-Decyltetradeceth-12, PPG-6-Decyltetradeceth-20, PPG-6-Decyltetradeceth-30, PPG-13-Decyltetradeceth-24, PPG-20-Decyltetradeceth-10, PPG-2-Isodeceth-4, PPG-2-Isodeceth-6, PPG-2-Isodeceth-8, PPG-2-Isodeceth-9, PPG-2-Isodeceth-10, PPG-2-Isodeceth-12, PPG-2-Isodeceth-18, PPG-2-Isodeceth-25, PPG-4-Isodeceth-10, PPG-12-Laneth-50, PPG-2-Laureth-5, PPG-2-Laureth-8, PPG-2-Laureth-12, PPG-3-Laureth-8, PPG-3-Laureth-9, PPG-3-Laureth-10, PPG-3-Laureth-12, PPG-4 Laureth-2, PPG-4 Laureth-5, PPG-4 Laureth-7, PPG-4-Laureth-15, PPG-5-Laureth-5, PPG-6-Laureth-3, PPG-25-Laureth-25, PPG-7 Lauryl Ether, PPG-3-Myreth-3, PPG-3-Myreth-11, PPG-20-PEG-20 Hydrogenated Lanolin, PPG-2-PEG-11 Hydrogenated Lauryl Alcohol Ether, PPG-12-PEG-50 Lanolin, PPG-12-PEG-65 Lanolin Oil, PPG-40-PEG-60 Lanolin Oil, PPG-1-PEG-9 Lauryl Glycol Ether, PPG-3-PEG-6 Oleyl Ether, PPG-23-Steareth-34, PPG-30 Steareth-4, PPG-34-Steareth-3, PPG-38 Steareth-6, PPG-1 Trideceth-6, PPG-4 Trideceth-6, and PPG-6 Trideceth-8.

Preferably, the alkoxylated fatty alcohol is selected from

Advantageously, the nonionic surfactant selected from alkoxylated fatty alcohols is present in the composition in an amount ranging from 0.1 wt. % to 10 wt. %, preferably from 0.2 wt. % to 5 wt. %, more preferably from 0.5 wt. % to 2 wt. %, relative to the total weight of the composition.

### Aqueous Phase

As an oil-in-water emulsion, the cosmetic composition of the present invention comprises a continuous aqueous phase.

Advantageously, said aqueous phase is present in an amount ranging from 50 wt. % to 95 wt. %, preferably from 55 wt. % to 90 wt. % of the total weight of the composition.

Said aqueous phase comprises water.

Advantageously, water is present in the composition of the present invention in an amount ranging from 20 wt. % to 90 wt. %, preferably from 50 wt. % to 85 wt. %, relative to the total weight of the composition.

Preferably, the continuous aqueous phase comprises an organic solvent miscible with water (at room temperature 25° C.) such as, ethanol, phenoxyethanol, glycol and polyols having from 2 to 20 carbon atoms, preferably from 2 to 10 carbon atoms, and preferentially having from 2 to 6 carbon atoms, such as glycerin, propylene glycol, butylene glycol, pentylene glycol, hexylene glycol, caprylyl glycol, dipropylene glycol, diethylene glycol; and mixtures thereof, so as to provide a hydration effect.

### Oily Phase

As an oil-in-water emulsion, the composition of the present invention comprises at least one dispersed oily phase.

Said oily phase comprises at least one oil.

The oil can be volatile or non-volatile.

The term “oil” means a water-immiscible non-aqueous compound that is liquid at room temperature (25° C.) and at atmospheric pressure (760 mmHg).

The term “non-volatile oil” means an oil that may remain on keratin materials at room temperature and atmospheric pressure for at least several hours and that especially has a vapour pressure of less than 10⁻³ mmHg (0.13 Pa). A non-volatile oil may also be defined as having an evaporation rate such that, under the conditions defined previously, the amount evaporated after 30 minutes is less than 0.07 mg/cm².

These oils may be of plant, mineral or synthetic origin.

Said oil can be selected from hydrocarbonated, silicone or fluorinated oils.

The term “hydrocarbon-based oil” means an oil formed essentially from, or even constituted by, carbon and hydrogen atoms, and optionally O and N atoms, and free of Si and F heteroatoms. Such oil can contain alcohol, ester, ether, carboxylic acid, amine and/or amide groups.

The term “silicone oil” means an oil containing at least one silicon atom, especially containing Si—O groups.

The term “fluorinated oil” means an oil containing at least one fluorine atom.

The oil can be, for example, present in an amount ranging from 0.01 wt. % to 40 wt. %, preferably from 0.05 wt. % to 30 wt. %, more preferably from 0.1 wt. % to 10 wt. %, relative to the total weight of the composition.

### Additional Cosmetic Active Ingredients

The composition of the present invention may comprise an additional cosmetic active ingredient in addition to the cosmetic active compound of formula (I) as defined previously.

As examples of cosmetic active ingredient, mention can be made of moisturizing agents such as protein hydrolysates, and PEG/PPG/POLYBUTYLENE GLYCOL-8/5/3 GLYCERIN; botanical extracts (such as carica papaya fruit extract, hydrolyzed opuntia ficus-indica flower extract); vitamins such as vitamin A (retinol), vitamin E (tocopherol), vitamin C (ascorbic acid), vitamin B5 (panthenol), vitamin B3 (niacinamide), and derivatives of said vitamins (in particular esters) and mixtures thereof; antiaging active ingredients (such as adenosine); antioxidant (such as salicylic acid, tocopherol); anti-acne ingredients (such as hydroxyethylpiperazine ethane sulfonic acid); urea; caffeine; yeast extract; monosaccharide (such as rhamnose); tightening agents; agents acting on the microcirculation, and mixtures thereof.

It is easy for the skilled in the art to adjust the amount of the additional cosmetic active ingredient based on the final use of the composition according to the present invention.

### Additional Adjuvants or Additives

The composition of the present invention may comprise may also contain conventional cosmetic adjuvants or additives, for instance fragrances, chelating agents (for example, phytic acid), preserving agents (for example, salicylic acid) and bactericides, thickeners (such as xanthan gum), hydrotropes (for example, acetyl trifluoromethylphenyl valylglycine); pH regulators (for example, triethanolamine, citric acid and sodium hydroxide), and mixtures thereof.

The skilled in the art can select the amount of the additional adjuvants or additive so as not to adversely impact the final use of the composition according to the present invention.

According to a particularly preferred embodiment, the present invention provides a composition in the form of an oil-in-water emulsion for caring for keratin materials comprising:
(i) from 0.6 wt. % to 10 wt. % of at least one cosmetic active compound selected from C-β-D-xylopyranoside-2-hydroxypropane, C-α-D-xylopyranoside-2-hydroxypropane, and a mixture thereof;
(ii) from 0.08 wt. % to 0.5 wt. % of at least one anionic surfactant selected from taurates;
(iii) from 0.25 wt. % to 2 wt. % of at least one nonionic surfactant selected from Polyglyceryl-5 laurate, Polyglyceryl-5 Myristate, Polyglyceryl-5 Oleate, Polyglyceryl-5 Stearate, Polyglyceryl-10 Caprate, Polyglyceryl-10 Laurate, Polyglyceryl-10 Myristate, Polyglyceryl-10 Oleate, Polyglyceryl-10 stearate, Polyglyceryl-10 Isostearate, and a mixture thereof; and
(iv) from 0.5 wt. % to 2 wt. % of at least one nonionic surfactant selected from ethoxylated/propoxylated C8-C24 alcohols with 1-50 ethylene oxide units and 1-50 propylene oxide units.

### Galenic Form and Method

The composition of the present invention is in the form of an oil-in-water emulsion. Preferably, the composition of the present invention is a microemulsion.

The “microemulsion” may be defined in two ways, namely, in a broader sense and in a narrower sense. That is to say, there are one case (“microemulsion in the narrow sense”) in which the microemulsion refers to a thermodynamically stable isotropic single liquid phase containing a ternary system having three ingredients of an oily component, an aqueous component and a surfactant, and the other case (“microemulsion in the broad sense”) in which among thermodynamically unstable typical emulsion systems the microemulsion additionally includes those such emulsions presenting transparent or translucent appearances due to their smaller particle sizes (Satoshi Tomomasa, et al., Oil Chemistry, Vol. 37, No. 11 (1988), pp. 48-53). The “microemulsion” as used herein refers to a “microemulsion in the broad sense”.

The composition of the present invention can be used for caring for keratin materials.

According to the second aspect, the present invention provides a non-therapeutic method for caring for keratin materials, comprising applying the composition according to the first aspect of the present invention to the keratin materials.

In particular, the keratin material is the skin.

The following examples serve to illustrate the present invention without, however, being limiting in nature.

### EXAMPLES

Main raw materials used, trade names and supplier thereof are listed in Table 1.

TABLE 1

| INCI | Trade Name | Manufacturer |
|---|---|---|
| HYDROXYPROPYL | MEXORYL SDL | CHIMEX (NOVEAL) |
| TETRAHYDROPYRANTRIOL | | |
| POLYGLYCERYL-5 LAURATE | SUNSOFT A-121E-C | TAIYO KAGAKU |
| SODIUM METHYL | NIKKOL SMT | NIKKO |
| STEAROYL TAURATE | | |
| PPG-6- | NIKKOL SG-DTD630 | NIPPON SURFACTANT |
| DECYLTETRADECETH-30 | | INDUSTRIES |
| ADENOSINE | ADENOSINE (L) | XINXIANG TUOXIN PHARM |
| ACETYL | MEXORYL SAR | CHIMEX (NOVEAL) |
| TRIFLUOROMETHYLPHENYL | | |
| VALYLGLYCINE | | |
| HYDROXYETHYLPIPERAZINE | HEPES-LUV | TAIWAN HOPAX |
| ETHANE SULFONIC ACID | | |
| YEAST EXTRACT | FIRMALIFT GRV | SILAB |
| ETHYLHEXYL PALMITATE | CEGESOFT ® C 24 | BSAF |
| ISOPROPYL LAUROYL | ELDEW SL-205 | AJINOMOTO |
| SARCOSINATE | | |
| XANTHAN GUM | KELTROL CG-T | CP KELCO |
| PEG/PPG/POLYBUTYLENE | WILBRIDE S-753L | NOF CORPORATION |
| GLYCOL-8/5/3 GLYCERIN | | |
| BIS-PEG-18 METHYL ETHER | DOWSIL ™ 2511 | DOW CORNING (DOW |
| DIMETHYL SILANE | COSMETIC WAX | CHEMICAL) |
| METHYL GLUCETH-10 | GLUCAM ™ E-10 | LUBRIZOL |
| | HUMECTANT | |
| MADECASSOSIDE | MADECASSOSIDE | SERDEX (SEPPIC) |

### Invention Example 1 and Comparative Examples 1-4

Compositions of invention example (IE) 1 and comparative examples (CE) 1-4 were prepared based on the amounts given in Table 2. The amounts are given in % by weight of active ingredient relative to the total weight of the composition.

TABLE 2

| INCI US | CE1 | CE2 | CE3 | CE4 | IE1 |
|---|---|---|---|---|---|
| DISODIUM EDTA | 0.1 | 0.05 | 0.05 | 0.05 | 0.05 |
| ADENOSINE | 0.1 | 0.04 | 0.04 | 0.04 | 0.04 |
| ACETYL | 0.1 | | 0.1 | | |
| TRIFLUOROMETHYLPHENYL | | | | 0.1 | 0.1 |
| VALYLGLYCINE | | | | | |
| HYDROXYETHYLPIPERAZINE | | 0.5 | 5 | 5 | 5 |
| ETHANE SULFONIC ACID | | | | | |
| YEAST EXTRACT | 1 | 1 | 1 | 1 | 1 |
| RHAMNOSE | | 0.1 | 0.1 | 0.1 | 0.1 |
| ETHYLHEXYL PALMITATE | 0.5 | 0.3 | 0.3 | 0.3 | 0.3 |
| ISOPROPYL LAUROYL | 0.5 | | | | |
| SARCOSINATE | | | | | |
| FRAGRANCE | 0.08 | 0.035 | 0.04 | 0.04 | 0.04 |
| XANTHAN GUM | 0.15 | 0.11 | 0.11 | 0.11 | 0.11 |
| PEG/PPG/POLYBUTYLENE | | 0.2 | 0.2 | 0.2 | 0.2 |
| GLYCOL-8/5/3 GLYCERIN | | | | | |
| SALICYLIC ACID | 0.2 | 0.2 | 0.2 | 0.2 | 0.2 |
| PHENOXYETHANOL | 0.25 | 0.5 | 0.5 | 0.5 | 0.5 |
| BIS-PEG-18 METHYL ETHER | 3.5 | | | | |
| DIMETHYL SILANE | | | | | |
| BUTYLENE GLYCOL | 5 | | 5 | | |
| ETHANOL | | 4 | 2 | 2 | 2 |
| GLYCERIN | 5 | 3 | 3 | 3 | 3 |
| PROPYLENE GLYCOL | | | | 0.625 | 0.625 |
| HYDROXYPROPYL | | | | | 0.875 |
| TETRAHYDROPYRANTRIOL | | | | | |
| METHYL GLUCETH-10 | 3 | | | | |
| PPG-6-DECYLTETRADECETH-30 | 1 | 1 | 1 | 1 | 1 |
| SODIUM METHYL STEAROYL | 0.2 | 0.2 | 0.1 | 0.1 | 0.1 |
| TAURATE | | | | | |
| POLYGLYCERYL-5 LAURATE | 1 | 0.3 | 0.3 | 0.3 | 0.3 |
| MADECASSOSIDE | 0.01 | | 0.01 | 0.01 | 0.01 |
| TOCOPHEROL | | 0.1 | 0.1 | 0.1 | 0.1 |
| WATER | QS100 | QS100 | QS100 | QS100 | QS100 |

Composition of invention example (IE) 1 represents composition according to the present invention.

Compositions of comparative examples 1-4 do not comprise any compound of formula (I).

### Preparation Process

The compositions listed above were prepared as follows, taking the composition of invention formula 1 as an example:
1) adding water and all raw materials of the aqueous phase into a beaker and heating to 75° C., meanwhile mixing to ensure all raw materials dissolve;
2) adding raw materials of the oily phase and mixing until the mixture becomes homogeneous;
3) adding the rest of raw materials one by one and mixing until a homogeneous composition was obtained.

### Evaluation

The stability of each composition prepared above was evaluated by observing the aspect of each composition immediately after it was prepared (0 day), 1 day after being still from −20° C. to 20° C. per 24 hours, and 10 days after being still from −20° C. to 20° C. per 24 hours.

If the composition tested becomes cloudy or opaque after being still for 1 day or 10 days from −20° C. to 20° C. per 24 hours, then the composition fails the stability test. If the composition tested remains transparent after being still for 10 days from −20° C. to 20° C. per 24 hours, then the composition passes the stability test.

The results of aspect and stability of each composition of invention example 1 and comparative examples 1-4 were listed in Table 3.

TABLE 3

| | Aspect | | | |
|---|---|---|---|---|
| Examples | 0 day | 1 day | 10 day | Stability |
| CE1 | Transparent | Opaque | NA | Fail |
| CE2 | Transparent | Opaque | NA | Fail |
| CE3 | Transparent | Transparent | Opaque | Fail |
| CE4 | Transparent | Transparent | Opaque | Fail |
| IE1 | Transparent | Transparent | Transparent | Pass |

NA: not observed.

It can be seen that the composition of invention example 1 can remain transparent after being still for 10 days from −20° C. to 20° C. per 24 hours, while compositions of comparative examples 1-2 are opaque when observed after being still for 1 day, and compositions of comparative examples 3-4 are opaque when observed after being still for 10 days.

1. A composition in the form of an oil-in-water emulsion for caring for keratin materials comprising:
(i) at least one cosmetic active compound selected from C-glycosides of formula (I) 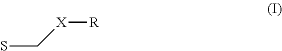
wherein
R represents a saturated C₁ to C₁₀, alkyl radical which may optionally be substituted by at least one radical selected from the group consisting of OH, COOH and COOR″₂, wherein R″₂ is a saturated C₁-C₄ radical,
S represents a monosaccharide or a polysaccharide comprising up to 20 sugar units in pyranose and/or furanose form and of the L and/or D series, optionally substituted by a hydroxyl group which is necessarily free and one or more optionally protected amine functional group(s), and
X represents a radical selected from the group consisting of —CO—, —CH(OH)—, —CH(NH₂)—, —CH(NHCH₂CH₂CH₂OH)—, —CH(NHPh)— and —CH(CH₃)— groups,
the S—CH₂—X bond represents a bond of C-anomeric nature, which can be α or β,
and also their physiologically acceptable salts, their solvates and their optical and geometrical isomers;
(ii) at least one anionic surfactant;
(iii) at least one non-ionic surfactant selected from alkoxylated alcohols; and
(iv) at least one non-ionic surfactant selected from fatty acid esters of polyglycerol.

2. The composition according to claim 1, wherein the cosmetic active compound is selected from the group consisting of
C-β-D-xylopyranoside-n-propane-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxypropane,
1-(C-α-D-glucopyranosyl)-2-hydroxypropane,
1-(C-β-D-galactopyranosyl)-2-hydroxypropane,
1-(C-α-D-galactopyranosyl)-2-hydroxypropane,
1-(C-β-D-fucofuranosyl)propan-2-one,
1-(C-α-D-fucofuranosyl)propan-2-one,
1-(C-β-L-fucofuranosyl)propan-2-one,
1-(C-α-L-fucofuranosyl)propan-2-one,
C-β-D-maltopyranoside-n-propane-2-one,
C-α-D-maltopyranoside-n-propan-2-one,
C-β-D-maltopyranoside-2-hydroxypropane,
C-α-D-maltopyranoside-2-hydroxypropane, and
isomers and mixtures thereof.

3. The composition according to claim 1, wherein the cosmetic active compound is present in an amount ranging from 0.5 wt. % to 15 wt %, relative to the total weight of the composition.

4. The composition according to claim 1, wherein the anionic surfactant is selected from the group consisting of taurates and mixtures thereof.

5. The composition according to claim 1, wherein the anionic surfactant is present in an amount ranging from 0.05 wt. % to 2 wt. %, relative to the total weight of the composition.

6. The composition according to claim 1, wherein the fatty acid ester of polyglycerol is selected from compounds of formula (II), 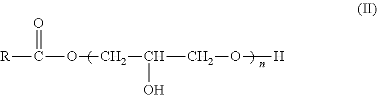
wherein:
R represents a linear or branched C₅-C₃₀ alkyl or alkenyl; and
n is an integer ranging from 5 to 30

7. The composition according to claim 1, wherein the fatty acid ester of polyglycerol is selected from the group consisting of Polyglyceryl-5 laurate, Polyglyceryl-5 Myristate, Polyglyceryl-5 Oleate, Polyglyceryl-5 Stearate, Polyglyceryl-10 Caprate, Polyglyceryl-10 Laurate, Polyglyceryl-10 Myristate, Polyglyceryl-10 Oleate, Polyglyceryl-10 stearate, Polyglyceryl-10 Isostearate, and a mixture thereof.

8. The composition according to claim 1, wherein the nonionic surfactant selected from fatty acid esters of polyglycerol is present in an amount ranging from 0.1 wt. % to 5 wt. %, relative to the total weight of the composition.

9. The composition according to claim 1, wherein the alkoxylated fatty alcohol is selected from ethers formed from the reaction of a fatty alcohol with ethylene oxide and/or propylene oxide.

10. The composition according to claim 1, wherein the alkoxylated fatty alcohol is ethoxylated/propoxylated C8-C30 alcohols with 1-50 ethylene oxide units and 1-50 propylene oxide units.

11. The composition according to claim 1, wherein the nonionic surfactant selected from alkoxylated fatty alcohols is present in an amount ranging from 0.1 wt. % to 10 wt. %, relative to the total weight of the composition.

12. The composition according to claim 1, comprising, relative to the total weight of the composition:
(i) from 0.6 wt. % to 3.5 wt. % of at least one cosmetic active compound selected from C-β-D-xylopyranoside-2-hydroxypropane, C-α-D-xylopyranoside-2-hydroxypropane, and a mixture thereof;
(ii) from 0.08 wt. % to 0.5 wt. % of at least one anionic surfactant selected from the group consisting of taurates;
(iii) from 0.25 wt. % to 2 wt. % of at least one nonionic surfactant selected from the group consisting of Polyglyceryl-5 laurate, Polyglyceryl-5 Myristate, Polyglyceryl-5 Oleate, Polyglyceryl-5 Stearate, Polyglyceryl-10 Caprate, Polyglyceryl-10 Laurate, Polyglyceryl-10 Myristate, Polyglyceryl-10 Oleate, Polyglyceryl-10 stearate, Polyglyceryl-10 Isostearate, and a mixture thereof; and
(iv) from 0.5 wt. % to 2 wt. % of at least one nonionic surfactant selected from the group consisting of ethoxylated/propoxylated C8-C24 alcohols with 1-50 ethylene oxide units and 1-50 propylene oxide units.

13. A non-therapeutic method for caring for keratin materials, comprising applying the composition according to claim 1 to the keratin materials.

14. The composition according to claim 1, wherein the anionic surfactant is selected from the group consisting of sodium salt of palm kernel oil methyltaurate, sodium N-cocoyl-N-methyltaurate, sodium palmitoyl methyltaurate, sodium methyl stearoyl taurate, and mixtures thereof.
